(19) United States
(12) Patent Application Publication
(10) Pub. No.: US 2015/0025371 A1
(43) Pub. Date: Jan. 22, 2015

(54) METHOD FOR DISPLAYING IMAGE DATA OF BODY REGIONS OF A PATIENT, THE BODY REGIONS BEING PRONE TO DEMENTIA, AND A MEDICAL IMAGING SYSTEM WHICH IS DESIGNED TO CARRY OUT THE METHOD

(71) Applicant: Siemens Aktiengesellschaft, Munich (DE)

(72) Inventors: Jens-Christoph GEORGI, Nuernberg (DE); Björn HEISMANN, Erlangen (DE); Sebastian SCHMIDT, Weisendorf (DE); Martin TASLER, Erlangen (DE)

(21) Appl. No.: 14/315,607

(22) Filed: Jun. 26, 2014

(30) Foreign Application Priority Data
Jul. 17, 2013 (DE) 102013214050.9

(51) Int. Cl. A61B 6/00; G06T 7/00; G06T 11/00; A61B 6/03; A61B 5/00

(52) U.S. Cl. CPC A61B 6/501; A61B 6/037; A61B 6/5247; A61B 6/481; A61B 5/4088; G06T 11/008; G06T 7/0079
USPC 600/427

(57) ABSTRACT

A method is disclosed for displaying image data of body regions of a patient using a medical imaging system including a first imaging apparatus and a positron emission tomography apparatus, the body regions being prone to dementia. The method includes provision of first image data recorded using the first imaging apparatus; provision of second image data recorded using the positron emission tomography apparatus, the first image data and the second image data being recorded simultaneously or at short intervals of time consecutively; segmentation of the first image data in respect of body regions prone to dementia, a segmentation mask being generated to this end on the basis of the first image data; generation of results data, the results data including a selection of voxels in the second image data made using the segmentation mask; and display of the results data.

### PRIORITY STATEMENT

The present application hereby claims priority under 35 U.S.C. §119 to German patent application number DE 102013214050.9 filed Jul. 17, 2013, the entire contents of which are hereby incorporated herein by reference.

### FIELD

At least one embodiment of the invention generally relates to a method and medical imaging system for displaying image data.

### BACKGROUND

Evidence of whether a patient is affected by dementia can be obtained using a positron emission tomography examination (PET examination), a radiopharmaceutical—for example Amyvid—being administered to the patient to this end. However, using this examination method may lead to falsifications of the results, since for example only deposits of the radiopharmaceutical in the gray brain matter are relevant. These deposits in the gray brain matter cannot however be displayed in isolation using a PET examination.

Moreover, from the recorded positron emission tomography data (PET data) alone, it is not possible to determine a volume of the gray brain matter, and based on the PET data it is not possible to segment the mapped gray brain matter, which would however be necessary in order to quantify amyloid plaques which are characteristic of dementia, in particular Alzheimer's disease. Furthermore, because of the low spatial resolution in the PET data, partial volume effects affect the result of the measurement.

### SUMMARY

At least one embodiment of the present invention is, in particular, directed to enabling regions of the brain prone to dementia to be displayed unambiguously in the positron emission tomography data. Advantageous embodiments are described in the subclaims.

At least one embodiment of the invention is directed to a method for displaying image data of body regions of a patient using a medical imaging system which has a first imaging apparatus and a positron emission tomography apparatus, the body regions being prone to dementia, the method including:

provision of first image data recorded using the first imaging apparatus,
provision of second image data recorded using the positron emission tomography apparatus, the first image data and the second image data being recorded simultaneously or at short intervals of time consecutively,
segmentation of the first image data in respect of body regions prone to dementia, a segmentation mask being generated on the basis of the first image data and
generation of results data, the results data comprising a selection of voxels in the second image data made using the segmentation mask and
display of the results data.

Furthermore, at least one embodiment of the invention is based on a medical imaging system with a first imaging apparatus, a positron emission tomography device and a computing unit, the medical imaging system being designed to carry out a method for displaying image data of body regions which are prone to dementia, the method comprising:

provision of first image data recorded using the first imaging apparatus,
provision of second image data recorded using the positron emission tomography apparatus, the first image data and the second image data being recorded simultaneously or at short intervals of time consecutively,
segmentation of the first image data in respect of body regions prone to dementia, a segmentation mask being generated to this end on the basis of the first image data,
generation of results data, the results data comprising a selection of voxels in the second image data made using the segmentation mask and
display of the results data.

Furthermore, at least one embodiment of the invention is based on a computer program which can be loaded directly into a memory of a programmable computing unit of the medical imaging system, having program means in order to execute a method for displaying image data of body regions which are prone to dementia, when the computer program is executed in the computing unit of the medical imaging system. Such implementation using software has the advantage that existing computing units of medical imaging systems can be modified by implementing the computer program appropriately in order inventively to enable, using a medical imaging system, the display of image data of body regions which are prone to dementia.

### BRIEF DESCRIPTION OF THE DRAWINGS

Further advantages, features and details of the invention will become apparent from the example embodiment described hereinbelow as well as with reference to the drawings,

in which:

FIG. 1 shows a schematic representation of a medical imaging system with a magnetic resonance apparatus and a positron emission tomography apparatus, and

FIG. 2 shows a flow chart of an embodiment of the inventive method for displaying image data of body regions of a patient which are prone to dementia.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the invention is directed to a method for displaying image data of body regions of a patient using a medical imaging system which has a first imaging apparatus and a positron emission tomography apparatus, the body regions being prone to dementia, the method including:

provision of first image data recorded using the first imaging apparatus,
provision of second image data recorded using the positron emission tomography apparatus, the first image data and the second image data being recorded simultaneously or at short intervals of time consecutively,
segmentation of the first image data in respect of body regions prone to dementia, a segmentation mask being generated on the basis of the first image data and
generation of results data, the results data comprising a selection of voxels in the second image data made using the segmentation mask and
display of the results data.

Thanks to the inventive embodiment, the second image data, in particular the positron emission tomography image data (PET image data), can advantageously be reduced to image regions and/or to voxels which map and/or comprise body regions of the patient which are prone to dementia, such as Alzheimer's disease for example. In this way the place from which the positron emission tomography signals (PET signals) recorded were emitted can be located particularly precisely and thus the PET signals recorded are unambiguously assigned to a body region of the patient during an assessment and/or diagnosis subsequent to the inventive method. In particular, the PET signals can be unambiguously assigned to body regions which are prone to dementia, thereby preventing an erroneous interpretation of the PET signals. The segmentation mask here preferably comprises an assignment of individual voxels in the image data to the body regions which are prone to dementia.

Particularly advantageously, the body regions which are prone to dementia, in particular Alzheimer's disease, here comprise gray brain matter of the patient. Moreover, provision can also be made for the segmentation mask to also contain a selection of voxels which map white brain matter in the image data, so that a comparison of signal strengths resulting from the gray brain matter and of signal strengths resulting from the white brain matter can also be generated. Preferably the segmentation of the voxels which map white brain matter in the image data takes place separately from the segmentation of voxels which map the gray brain matter in the image data.

In this connection, provision of image data should in particular be understood as loading stored image data from a storage unit and/or capturing or recording the image data in the form of raw imaging data using the first imaging apparatus and/or the positron emission tomography apparatus (PET apparatus). Furthermore, segmentation should in particular be understood as a selection of image elements and/or voxels in the first image data, the selection being made on the basis of an anatomical structure of the patient displayed in the first image data. The segmentation can take place using segmentation algorithms, for example a region-growing method or a threshold value method, etc.

Furthermore, a segmentation mask should in particular be understood as a segmentation instruction which lays down which of the image elements and/or voxels in the image data are selected. Preferably here the first imaging apparatus is designed to record data with a high spatial resolution and/or a high contrast ratio in the recorded image data, so that an advantageous segmentation mask can be provided which is essentially restricted to the image regions and/or voxels which map body regions and/or organs of the patient which are prone to dementia. In particular, the segmentation takes place on the basis of a differentiation of gray brain matter and white brain matter in the first image data.

Furthermore, a brief time interval at which the recording of the first raw imaging data and the recording of the second raw imaging data take place consecutively, in particular a maximum time interval of 20 minutes, should more advantageously be understood as a maximum time interval of ten minutes and particularly preferably a maximum time interval of five minutes, so that for both imaging examinations the patient assumes the same examination position in respect of detector components of the various imaging apparatuses.

Furthermore, it is proposed that the first imaging apparatus comprises a magnetic resonance apparatus, which means a particularly advantageous high spatial resolution and/or a high contrast ratio can be achieved in the image data of the examined body region of the patient. Associated with this, for example, an advantageous localization of the signals in the positron emission tomography data can be achieved on the basis of the spatial information of the magnetic resonance image data. Moreover, using the image data recorded by the magnetic resonance apparatus, an advantageous display of organs and soft tissues of patients can be achieved.

The recording of magnetic resonance data for recording and/or displaying the examined body region of the patient can here take place during and/or immediately after the administration of a medical indicator agent, in particular a magnetic resonance contrast agent such as Gd-DTPA (Gadopentetate Dimeglumine), for example. Preferably the recording of the magnetic resonance data uses a magnetic resonance sequence which generates a high spatial resolution and/or an advantageous gray/white contrast in the image data, so that in particular isotrope voxels are present in the image data. The magnetic resonance sequence for example comprises a STIR sequence, an MPRAGE as a 3D sequence, etc. An echo time and/or a relaxation time of the magnetic resonance sequence is here preferably selected and/or set such that in particular a contrast between white brain matter and gray brain matter is maximized in the magnetic resonance data recorded. To this end test measurements can also be made before the image data is captured.

For example, for an examination as to whether a patient is suffering from dementia, such as Alzheimer's disease for example, only the gray brain matter of the patient is examined using a PET examination, since senile plaques and/or fibrillar deposits form in the gray brain matter in particular when Alzheimer's disease is present, the accumulation of which in the gray brain matter can be recorded and/or determined using the PET examination. A restriction to voxels which map the gray brain matter in the PET image data can be provided particularly easily if the results data is restricted solely to the selection of voxels in the second image data made using the segmentation mask.

In an advantageous development of an embodiment of the invention, it is proposed that the recording of the second image data provided takes place using the positron emission tomography apparatus following an administration of a radiopharmaceutical, the radiopharmaceutical comprising an amyloid-plaque-binding radiopharmaceutical. An advantageous accumulation of the radiopharmaceutical on deposits comprising amyloid-beta deposits formed in the brain by dementia, in particular by Alzheimer's disease, can be achieved, and thus the deposits can be advantageously displayed in the PET data. Particularly advantageously the radiopharmaceutical comprises florbetapir (Amyvid).

A particularly simple and direct transfer of a segmentation mask generated using the first image data to the second image data can be achieved if the first image data and the second image data are based on the same coordinates system. Advantageously to this end the first and the second image data are recorded using a combined imaging system, so that a shared registration of the first and the second image data takes place. When the first image data and the second image data are based on different coordinates systems, an adjustment and/or a comparison between the first and the second image data in respect of conformity and/or alignment of the two coordinates systems can take place in the inventive method.

In a further embodiment of the invention, it is proposed that the generation of the results data comprises a quantitative evaluation of the second image data, which means that the degree of a disease can particularly advantageously be concluded on the basis of the results data during a subsequent assessment. For the quantitative evaluation in particular an activity rate and/or a standardized uptake value (SUV) is determined from the PET data, the activity rate and/or the SUV depending on accumulations of the radiopharmaceutical at deposits formed in the brain as a result of dementia, in particular Alzheimer's disease. Preferably the SUV comprises a physiological quantification of local concentrations of radioactivity. Included in the SUV is a metabolic process between the radiopharmaceutical used for the positron emission tomography examination and the body region examined, in particular a tumor region and/or a lesion. Here the SUV essentially represents a concentration of radioactivity in relation to an applied radioactivity.

Furthermore, it is proposed that the quantitative evaluation of the second image data comprises a correction of partial volume effects and/or a movement correction. The relevance of the quantitative evaluation and/or of the second results data can be advantageously increased here, and in particular a misinterpretation of the results data can be prevented. Partial volume effects occur in particular if the voxels of the second image data are larger than a structure of the body region of the patient to be mapped.

For example, a voxel of the second image data only partially maps the gray brain matter of the patient and another part of the voxel maps further regions of the brain, in particular white brain matter. In the data evaluation this voxel would be assigned in full to the gray brain matter, which would however result in considerable inaccuracies in the results data, since in the data evaluation an SUV for the assumption, on which the data evaluation is based, of a volume of the gray brain matter would be too small. Similarly too, when the patient moves, coverage of the segmentation mask with the selected body regions of the patient, in particular of the gray brain matter of the patient, is no longer complete, so that this too may result in inaccuracies in the results data.

Particularly advantageously, the correction of partial volume effects and/or the movement correction takes place using the first image data. This means a high spatial resolution in the first image data, which is greater than a spatial resolution in the second image data, and/or an advantageous contrast ratio and/or a mapping definition in the first image data can be employed and/or used for the correction of the results data. Thus, for example using the first image data a portion of the voxels in the second image data can be determined which does not map the selected body region, in particular the gray brain matter, and a corresponding proportional factor of the gray brain matter in the selected voxels can be taken into account when determining the results data.

Moreover, an ongoing recording and/or evaluation of the first image data allows a movement and/or change in position of the patient to be recorded and to be taken into account when calculating the results data. Particularly advantageously, this can be achieved using a combined medical imaging system with a magnetic resonance apparatus and an integrated positron emission tomography apparatus, since here the magnetic resonance measurement can take place in parallel to the positron emission tomography measurement without the patient thereby experiencing a change in position. Moreover, the different image data is here based on the same coordinates system.

In an advantageous development of an embodiment of the invention, it is proposed that the results data be evaluated in a further evaluation step in respect of a patient parameter and/or a parameter of a radiopharmaceutical administered. This means a patient-related evaluation of the results data can be achieved which is high in relevance. The parameter of the radiopharmaceutical administered preferably comprises an applied dose of the radiopharmaceutical. The patient parameter may for example be a body weight and/or a distribution space of the radiopharmaceutical and/or a body surface and/or a fat-free body mass, it being possible for the patient parameter to also depend on pharmacokinetics of the radiopharmaceutical. The pharmacokinetics here take into account as much as possible a totality of all processes which a radiopharmaceutical applied to the patient undergoes in the body of the patient. Included among these processes are intake of the radiopharmaceutical, distribution of the radiopharmaceutical in the body, breakdown of the radiopharmaceutical and excretion of the radiopharmaceutical.

Furthermore, at least one embodiment of the invention is based on a medical imaging system with a first imaging apparatus, a positron emission tomography device and a computing unit, the medical imaging system being designed to carry out a method for displaying image data of body regions which are prone to dementia, the method comprising:

provision of first image data recorded using the first imaging apparatus,
provision of second image data recorded using the positron emission tomography apparatus, the first image data and the second image data being recorded simultaneously or at short intervals of time consecutively,
segmentation of the first image data in respect of body regions prone to dementia, a segmentation mask being generated to this end on the basis of the first image data,
generation of results data, the results data comprising a selection of voxels in the second image data made using the segmentation mask and
display of the results data.

The second image data, in particular the PET image data, can advantageously be reduced to image regions and/or voxels, the image region and/or voxels mapping and/or comprising body regions of the patient which are prone to dementia, such as Alzheimer's disease for example. In particular in this way the place from which the PET signals recorded were emitted can be located particularly precisely and thus an unambiguous assignment of the PET signals recorded to a body region of the patient takes place during an assessment and/or diagnosis subsequent to the inventive method. Particularly advantageously, the positron emission tomography apparatus is here integrated inside the magnetic resonance apparatus, since the magnetic resonance measurement can here take place in parallel to the positron emission tomography measurement without the patient hereby experiencing a change of position.

Furthermore, at least one embodiment of the invention is based on a computer program which can be loaded directly into a memory of a programmable computing unit of the medical imaging system, having program means in order to execute a method for displaying image data of body regions which are prone to dementia, when the computer program is executed in the computing unit of the medical imaging system. Such implementation using software has the advantage that existing computing units of medical imaging systems can be modified by implementing the computer program appropriately in order inventively to enable, using a medical imaging system, the display of image data of body regions which are prone to dementia.

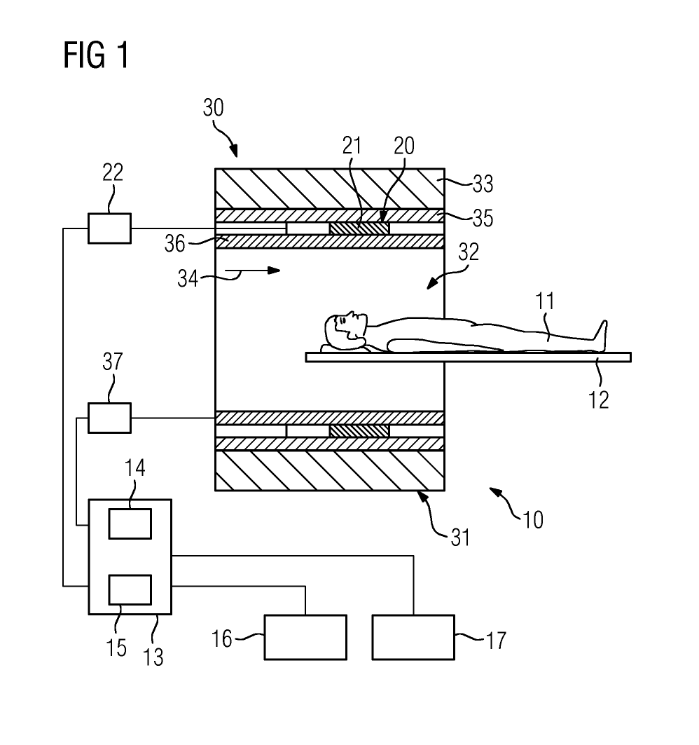

FIG. 1 shows a schematic representation of an embodiment of an inventive medical imaging system 10. The medical imaging system 10 comprises a combined imaging system which comprises a first imaging apparatus and a second imaging apparatus. The first imaging apparatus is formed in the present exemplary embodiment by a magnetic resonance apparatus 30. The second imaging apparatus is formed by a positron emission tomography apparatus 20 (PET apparatus 20). Also conceivable in principle is an embodiment of the first imaging apparatus as a computed tomography apparatus, etc. which can be combined with the PET apparatus 20.

The magnetic resonance apparatus 30 of the medical imaging system 10 comprises a magnet unit 31. The magnet unit 31 surrounds a patient receiving region 32 for receiving a patient 11, the patient receiving region 32 being surrounded cylindrically in a circumferential direction by the magnet unit 31. The patient 11 can be introduced into the patient receiving region 32 using a patient positioning apparatus 12 of the magnetic imaging system 10. For this purpose the patient positioning apparatus 12 is arranged so as to be movable within the patient receiving region 32.

The magnet unit 31 comprises a main magnet 33 which during operation of the magnetic resonance apparatus 30 is designed to generate a strong and in particular constant main magnetic field 34. The magnet unit 31 additionally has a gradient coil unit 35 for generating magnetic field gradients, which is used for spatial encoding during an imaging session. Moreover, the magnet unit 31 also has a high-frequency antenna unit 36 which serves to induce a polarization which arises in the main magnetic field 34 generated by the main magnet 33.

For the purpose of controlling the main magnet 33 of the gradient control unit 35 and of controlling the high-frequency antenna unit 36, the magnetic resonance apparatus 30 has a control unit 37 formed by a computing unit. The control unit 37 is used for central control of the magnetic resonance apparatus 30, such as performing a predetermined imaging gradient echo sequence for example. To this end the control unit 37 comprises a gradient control unit (not shown in greater detail) and a high-frequency antenna control unit (not shown in greater detail). Moreover, the control unit 37 comprises an evaluation unit (not shown in greater detail) for evaluating magnetic resonance image data.

The magnetic resonance apparatus 30 shown can obviously comprise further components that magnetic resonance apparatuses typically include. Moreover, the general mode of operation of a magnetic resonance apparatus 30 is known to the person skilled in the art, so a detailed description of the general components will be dispensed with.

The PET apparatus 20 of the medical imaging system 10 comprises several positron emission tomography detector modules 21 (PET detector modules 21) which are arranged in the form of a ring and surround the patient receiving region 32 in the circumferential direction. The PET detector modules 21 are here arranged between the high-frequency antenna unit 36 and the gradient coil unit 35 of the magnetic resonance apparatus 30 and thus are integrated in a particularly space-saving manner inside the magnetic resonance apparatus 30.

The PET detector modules 21 each have several positron emission tomography detector elements (PET detector elements) (not shown in greater detail) which are arranged to form a PET detector array which comprises a scintillation detector array containing scintillation crystals, for example LSO crystals. Furthermore, the PET detector modules 21 each comprise a photodiode array, for example avalanche photodiode array or APD photodiode array, which are arranged downstream of the scintillation detector array inside the PET detector modules 21. Moreover, the PET detector array has detector electronics (not shown in greater detail) which comprise an electrical amplifier circuit and other electronic components (not shown in greater detail).

To control the PET detector modules 21 the PET apparatus 20 has a control unit 22. The PET apparatus 20 shown can obviously comprise further components that PET apparatuses typically include. Moreover, the general mode of operation of a PET apparatus 20 is known to the person skilled in the art, so a detailed description of the general components will be dispensed with.

Using the PET detector modules 21 pairs of photons resulting from the annihilation of a positron with an electron are detected. Trajectories of the two photons encompass an angle of 180°. Moreover, both the photons each have an energy of 511 keV. The positron is here emitted by a radiopharmaceutical, the radiopharmaceutical being administered to the patient 11 by way of an injection. When penetrating matter the photons produced by the annihilation may be absorbed, the probability of absorption depending on the path length through the matter and the corresponding absorption coefficient of the matter.

Moreover, the medical imaging system 10 has a central computing unit 13 which for example coordinates recording of magnetic resonance image data using the magnetic resonance apparatus 30 and recording of PET image data using the PET apparatus 20 to one another for shared data recording. Moreover, the computing unit 13 comprises an evaluation unit (not shown in greater detail). The computing unit 13 further comprises a processor unit 14 and a storage unit 15. Control information such as imaging parameters, for example, as well as reconstructed image data, can be displayed on a display unit 16, for example on at least one monitor, of the medical imaging system 10 for viewing by an operator. Moreover, the medical imaging system 10 has an input unit 17 by means of which information and/or parameters can be entered by an operator during a measurement procedure.

The medical imaging system 10 shown can obviously comprise further components that medical imaging systems typically include. Furthermore, the general mode of operation of a medical imaging system 10 is known to the person skilled in the art, so a detailed description of the general components will be dispensed with.

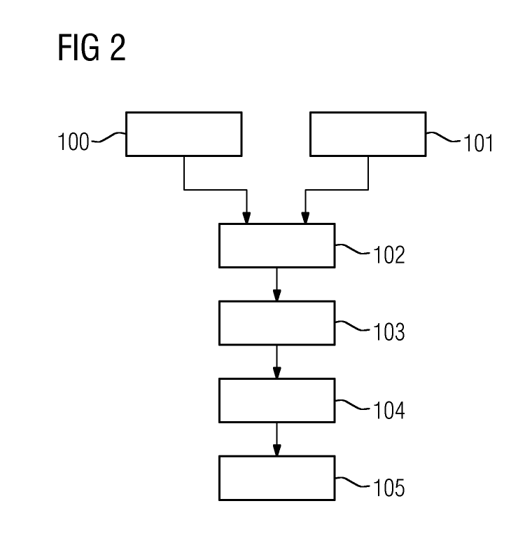

FIG. 2 schematically shows a flow chart of an embodiment of an inventive method for displaying image data of body regions of a patient which are prone to dementia. The present method concentrates in particular on body regions which are prone to Alzheimer's disease. In principle the method can also be applied to other types of dementia which may differ from Alzheimer's disease.

In a first method step 100 first image data formed from magnetic resonance image data and recorded using the magnetic resonance apparatus 30 is provided by the computing unit 13 from the body region of the patient 11 for further evaluation. The provision of the magnetic resonance image data in the first method step 100 comprises retrieving previously stored magnetic resonance image data from the storage unit 15 or also recording and/or capturing the magnetic resonance image data in the form of raw magnetic resonance data using the magnetic resonance apparatus 30. Moreover, it can also be provided for the recording of the provided magnetic resonance image data to take place during and/or after the administration of a medical indicator agent which is formed by a magnetic resonance contrast agent, for example Gd-DTPA.

Preferably the recording of the magnetic resonance image data uses a magnetic resonance sequence with a high spatial resolution and/or an advantageous high gray/white contrast, which in particular generate isotrope voxels in the image data. The magnetic resonance sequence for example comprises a STIR sequence, an MPRAGE as a 3D sequence and/or other sequences which appear expedient to the person skilled in the art. An echo time and/or a relaxation time of the selected magnetic resonance sequence is here preferably selected and/or set such that in particular a contrast between white brain matter and gray brain matter is maximized in the recorded magnetic resonance image data, so that the gray brain matter which is prone to Alzheimer's disease can be advantageously differentiated from the white brain matter. To this end test measurements can also be made before the magnetic resonance image data is captured. Setting of the echo time and/or the relaxation time can here take place at least in part automatically using the computing unit 13 or can also be entered manually by an operator.

In an embodiment of a further method, step 101 second image data formed from positron emission tomography image data (PET image data) and recorded using the PET apparatus 20 is provided by the computing unit 13 from the body region of the patient 11 for further evaluation. The body region of the patient 11 mapped using the PET image data is here the same body region of the patient 11 which is mapped using the magnetic resonance image data. The provision of the PET image data in the further method step 101 comprises retrieving previously stored image data from the storage unit 15 or also recording and/or capturing the PET image data of the PET apparatus 20.

The recording of the PET image data provided using the PET apparatus 20 takes place during and/or after the administration of a medical indicator agent which is formed by a radiopharmaceutical, the radiopharmaceutical comprising an amyloid-plaque-binding radiopharmaceutical, in particular Amyvid. Amyloid-plaque-binding radiopharmaceuticals, in particular Amyvid, preferably accumulate in the deposits in the gray brain matter caused by Alzheimer's disease, so that using this radiopharmaceutical a concentration of these deposits of the patient's Alzheimer's disease can be displayed in the PET image data. The recording of the PET image data can begin up to 90 minutes after the administration of the radiopharmaceutical.

The magnetic resonance image data has a higher spatial resolution than the PET image data. Moreover, the magnetic resonance image data has an advantageous contrast ratio, so that it is possible to differentiate between gray brain matter and white brain matter using the magnetic resonance image data and thus using the magnetic resonance image data it is possible to assign PET signals in the PET image data to a point of origin in the human body particularly unambiguously during an evaluation of the PET image data.

The recording of the provided magnetic resonance image data using the magnetic resonance apparatus 30 and the recording of the provided PET image data using the PET apparatus 20 take place simultaneously. Alternatively, the recording of the first magnetic resonance image data provided and the recording of the first PET image data provided can also take place at short time intervals consecutively, the short time interval between the recording of the first magnetic resonance image data and the first PET image data being a maximum time interval of 20 minutes, advantageously a maximum of ten minutes and particularly preferably a maximum of five minutes.

Moreover, the magnetic resonance image data and the PET image data are based on the same coordinates system. Individual voxels with the same coordinates in the magnetic resonance image data and in the PET image data here map the same body region of the patient 11. The magnetic resonance image data and the PET image data have the same coordinates within the coordinates system of the medical imaging system 10. When the magnetic resonance image data and the PET image data are based on different coordinates systems, an adjustment and/or a comparison between the magnetic resonance image data and the PET image data in respect of conformity and/or alignment of the two coordinates systems can also be effected in the method step 101 by the computing unit 13.

After the provision of the magnetic resonance image data and the PET image data the magnetic resonance image data is segmented by the computing unit 13, in a further method step 102 in respect of body regions which may be affected by Alzheimer's disease. Based on the magnetic resonance image data a segmentation mask is here generated by the computing unit 13, and contains a selection of voxels which map the body regions of the patient 11 which may be affected by Alzheimer's disease. To generate the segmentation mask, the computing unit 13 selects, in the method step 102, those voxels in the magnetic resonance image data which comprise the gray brain matter, the selection taking place on the basis of a differentiation between the gray brain matter and the white brain matter, so that the selected and/or segmented data contains only those image voxels in the magnetic resonance image data which map gray brain matter.

Furthermore, in the method step 102 the computing unit 13 can also effect a segmentation to select the voxels which map white brain matter in the magnetic resonance image data. However, this is only important if the radiopharmaceutical used to record or capture the PET image data also accumulates or is stored in the white brain matter, so that the PET image data also contains signals from the white brain matter. The segmentation of the voxels which map the white brain matter in the magnetic resonance image data however takes place separately from the segmentation of voxels which map the gray brain matter in the magnetic resonance image data. Here too the computing unit 13 generates a segmentation mask in the method step 102.

To segment the magnetic resonance image data, the computing unit 13 uses segmentation algorithms, for example a region-growing method and/or a threshold value method and/or other segmentation methods which seem expedient to the person skilled in the art.

Then in another method step 103, the computing unit 13 generates results data, the computing unit 13 to this end applying and/or transferring the segmentation mask to the PET image data, so that the results data comprises solely the selection of voxels in the PET image data made using the segmentation mask. The generation of the results data in the further method step 103 moreover comprises a quantitative evaluation of the PET image data. For the quantitative evaluation the computing unit 13 in particular determines an activity rate and/or a standardized uptake value (SUV) from the PET data, the activity rate and/or the SUV depending on accumulations of the radiopharmaceutical at deposits formed in the brain, in particular in the gray brain matter, as a result of Alzheimer's disease.

Furthermore, during the generation of the results data, in particular the quantitative evaluation of the PET image data, the computing unit 13 in the method step 103 undertakes a correction of partial volume effects and/or a movement correction. A correction of the partial volume effects is applied in particular in the case of voxels of the PET image data which are larger than a structure of the body region to be mapped, in particular the gray brain matter, of the patient 11.

If, for example, voxels of the PET image data only map part of the gray brain matter of the patient 11 and another part of the voxel maps further brain regions, in particular white brain matter, of the patient 11, the computing unit 13 determines a proportional factor of the gray brain matter within the voxel and this proportional factor is taken into account by the computing unit 13 when determining the results data. The proportional factor in the voxels of the PET image data of gray brain matter is determined by the computing unit 13 in the method step 103, using the magnetic resonance image data, since the magnetic resonance image data has a higher spatial resolution compared to the PET image data.

Similarly too, when the patient 11 moves, coverage of the segmentation mask with the selected body regions of the patient 11, in particular of the gray brain matter of the patient 11, is no longer complete, so that this too may result in inaccuracies in the results data. A corresponding movement correction is here taken into account by the computing unit 13 in the method step 104 of the generation of the results data. A corresponding correction parameter for the movement correction is determined by the computing unit 13 from the magnetic resonance image data in the method step 103, since on the basis of the magnetic resonance data an exact location of the patient 11 can be recorded.

When the radiopharmaceutical used to record or capture the PET image data also accumulates or is stored in the white brain matter, so that the PET image data also contains signals from the white brain matter, the partial volume effects in the method step 103 can also be undertaken by the computing unit 13 on the basis of PET signals which are assigned to the white brain matter. However, to this end, as already explained in greater detail in the method step 102, a segmentation of the image data in respect of the white brain matter must likewise have taken place.

In a further method step 104, the computing unit 13 evaluates the results data in respect of a patient parameter and/or a parameter of the radiopharmaceutical administered. The patient parameter may for example be a body weight and/or a distribution space of the radiopharmaceutical and/or a body surface and/or a fat-free body mass, it being possible for the patient parameter to also depend on pharmacokinetics of the radiopharmaceutical. The parameter of the radiopharmaceutical administered preferably comprises an applied dose of the radiopharmaceutical.

In a subsequent method step 105, the computing unit 13 is used to generate an optical display and/or output for the determined, corrected and evaluated results data, which is displayed for medical operating personnel using the display unit 16.

The method steps 100 to 105 are executed by the computing unit 13 together with the magnetic resonance apparatus 30 and the PET apparatus 20. To this end, the computing unit 13 comprises the requisite software and/or computer programs, which are stored in the storage unit 15. The software and/or computer programs comprise program segments/modules/etc. which are designed to execute described methods for evaluating magnetic resonance image data from the first imaging examination and PET image data from a second imaging examination on the patient 11, when the computer program and/or the software is executed in the computing unit 13 using the processor unit 14 of the medical imaging apparatus 10.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in detail on the basis of the preferred exemplary embodiment, the invention is not limited by the disclosed examples and other variations can be derived herefrom by the person skilled in the art, without departing from the scope of protection of the invention.

What is claimed is:

1. A method for displaying image data of body regions of a patient using a medical imaging system including a first imaging apparatus and a positron emission tomography apparatus, the body regions being prone to dementia, the method comprising:
provisioning first image data recorded using the first imaging apparatus;
provisioning second image data recorded using the positron emission tomography apparatus, the first image data and the second image data being recorded simultaneously or at short intervals of time consecutively;
segmenting the first image data in respect of body regions prone to dementia, a segmentation mask being generated based upon the first image data;
generating results data, the results data comprising a selection of voxels in the second image data made using the segmentation mask; and
displaying the generated results data.

2. The method of claim 1, wherein the body regions which are prone to dementia comprise a gray brain matter of the patient.

3. The method of claim 1, wherein the first imaging apparatus comprises a magnetic resonance apparatus.

4. The method of claim 1, wherein the results data is restricted solely to the selection of voxels in the second image data made using the segmentation mask.

5. The method of claim 1, wherein the recording of the second image data provided takes place using the positron emission tomography apparatus following an administration of a radiopharmaceutical, the radiopharmaceutical comprising an amyloid-plaque-binding radiopharmaceutical.

6. The method of claim 1, wherein the first image data and the second image data are based on the same coordinates system.

7. The method of claim 1, wherein the generation of the results data comprises a quantitative evaluation of the second image data.

8. The method of claim 7, wherein the quantitative evaluation of the second image data comprises at least one of a correction of partial volume effects and a movement correction.

9. The method of claim 8, wherein the at least one of the correction of partial volume effects and the movement correction takes place using the first image data.

10. The method of claim 1, wherein the results data is evaluated in a further evaluation step in respect of at least one of a patient parameter and a parameter of a radiopharmaceutical administered.

11. A medical imaging system, comprising:
a first imaging apparatus;
a positron emission tomography apparatus; and
a computing unit, the medical imaging system being designed to carry out the method of claim 1.

12. A computer program, directly loadable into a memory of a programmable computing unit of a medical imaging system, including program segments to execute the method of claim 1, when the computer program is executed in the computing unit of the medical imaging system.

13. The method of claim 2, wherein the first imaging apparatus comprises a magnetic resonance apparatus.

14. The method of claim 2, wherein the results data is restricted solely to the selection of voxels in the second image data made using the segmentation mask.

15. The method of claim 2, wherein the recording of the second image data provided takes place using the positron emission tomography apparatus following an administration of a radiopharmaceutical, the radiopharmaceutical comprising an amyloid-plaque-binding radiopharmaceutical.

16. The method of claim 2, wherein the first image data and the second image data are based on the same coordinates system.

17. The method of claim 2, wherein the generation of the results data comprises a quantitative evaluation of the second image data.

18. A medical imaging system, comprising:
a first imaging apparatus;
a positron emission tomography apparatus; and
a computing unit, the medical imaging system being designed to carry out the method of claim 2.

19. A computer readable medium including program code segments for, when executed on a control device of a radar system, causing the control device of the radar system to implement the method of claim 1.

20. A computer readable medium including program code segments for, when executed on a control device of a radar system, causing the control device of the radar system to implement the method of claim 2.
